(19) United States
(12) Plant Patent Application Publication
(10) Pub. No.: US 2023/0301219 P1
(43) Pub. Date: Sep. 21, 2023

(54) Cherry Tree Named "Himari"

(50) ;

(71) Applicant: Local Independent Administrative Agency Hokkaido Research Organization, Sapporo (JP)

(72) Inventors: Masayuki Yoshida, Hokkaido (JP); Mitsunobu Ikenaga, Hokkaido (JP); Tetsuya Inoue, Sapporo (JP); Tetsushi Uchida, Hokkaido (JP); Takuya Hirama, Hokkaido (JP); Yutaka Inagawa, Hokkaido (JP); Hiroshi Muramatsu, Hokkaido (JP)

(21) Appl. No.: 18/445,020

(22) Filed: Mar. 3, 2023

(30) Foreign Application Priority Data
Mar. 16, 2022 (JP) 36084

(51) Int. Cl. A01H 6/74

(52) U.S. Cl. PLT/181

(57) ABSTRACT

‘Himari’ is new cherry tree variety having excellent fruit-setting property and cold hardiness, a large fruit size, and large productivity. ‘Himari’ can be distinguished from similar varieties by its dark red color, medium to firm fruit, small stone size and large fruit size.

### CROSS REFERENCE TO RELATED APPLICATIONS

This application claims priority to Japanese Plant Variety Protection Application No. 36084, filed Mar. 16, 2022.

Genus and species: The cherry tree variety of this invention is botanically identified as Prunus avium (L.) L.

Variety denomination: The variety denomination is ‘Himari’.

### BACKGROUND OF THE INVENTION

The cherry breeding experiment that led to the selection of this variety started in Hokkaido, Japan in 1990 and has been carried out since 1992 in order to obtain a cherry variety having good eating quality, large size, good coloration, and cross compatibility with ‘Sato Nishiki’.

Cherry ‘Himari’ was created in 2002 by artificial crossing ‘Nanyo’ (which was obtained from a cross of ‘Napolean’ and an unknow variety) having a large size and excellent eating quality bred in Yamagata Prefecture as a seed parent and ‘Benitemari’ (which was obtained from a cross of ‘Bic’ and ‘Sato Nishiki’) having a large size and excellent eating quality and coloration also bred in Yamagata Prefecture as a pollen parent. In 2002, 467 seeds obtained by crossing were sowed and 264 individuals were obtained as of December 2002. After overwintering, 27 individuals surviving in 2003 were planted and investigated. Individual ‘60-18’ (also referred to as ‘HC10’ for further testing and later provided the variety designation of ‘Himari’) having excellent fruit-setting property and cold hardiness, a large fruit size, and large productivity was selected in 2013. This individual has excellent appearance and eating quality and had been subjected to a fruit regional adaptability test since 2014 in Yoichi town, Fukagawa City and further characterized for development since 2015 in Mashike Town. ‘Himari’ can be distinguished from its parents and other cherry tree varieties based on the collective distinctive combination of characteristics. The S gene of ‘Himari’ is S₁S₃ whereas the S gene of ‘Benitemari’ is S₁S_{6.} The flowering period for ‘Himari’ is 2 days later than ‘Sat Nishiki’, which is 1-2 days later than ‘Benitemari’. The ripening period in Hokkaido for ‘Himari’ is mid-late July in comparison to the ripening period for ‘Benitemari’, which is late July. ‘Benitemari’ has short, heart-shape oblate round fruit whereas ‘Himari’ has kidney-shaped fruit. The initial and subsequent asexual propagation confirmed that the new variety is stable and the progeny remain true to type.

### SUMMARY OF INVENTION

‘Himari’ is a late-maturing line which is harvested in mid- to late-July in Hokkaido. Compared to ‘Nanyo’, ‘Himari’ has a large size, good skin coloration, firm flesh, and excellent eating quality. The S genotype is S₁S_{3,} which is cross-compatible with most cultivated varieties including ‘Sato Nishiki’. ‘Himari’ can thus improve the pollination environment of orchards and contribute to ensuring the fruiting of other varieties.

‘Himari’ can be distinguished from similar varieties ‘Nanyo’ and ‘Sato Nishiki’ based on the color of skin, fruit firmness, and the size of the fruit and stone. The skin color of ‘Himari’ is “dark red”, compared to “light red” for ‘Nanyo’ and “red” for ‘Sato Nishiki’. The fruit firmness of ‘Himari’ is medium to firm compared to soft to medium firmness for ‘Nanyo’ and medium firmness for ‘Sato Nishiki’. The stone size of ‘Himari’ is small (0.32 g) compared to the stone of ‘Nanyo’, which is small to medium (0.35 g). The fruit of ‘Himari’ is very large (10.7 g) compared to medium-sized fruit of ‘Sato Nihsiki’ (7.6 g).

### BRIEF DESCRIPTION OF THE PHOTOGRAPHS

The colors in the photograph are depicted as nearly true as is reasonably possible to obtain in color reproductions of this type.

FIG. 1 illustrates a typical 9-year old ‘Himari’ tree (Naganuma-cho, Yubari-gun, Hokkaido) in July 2021.

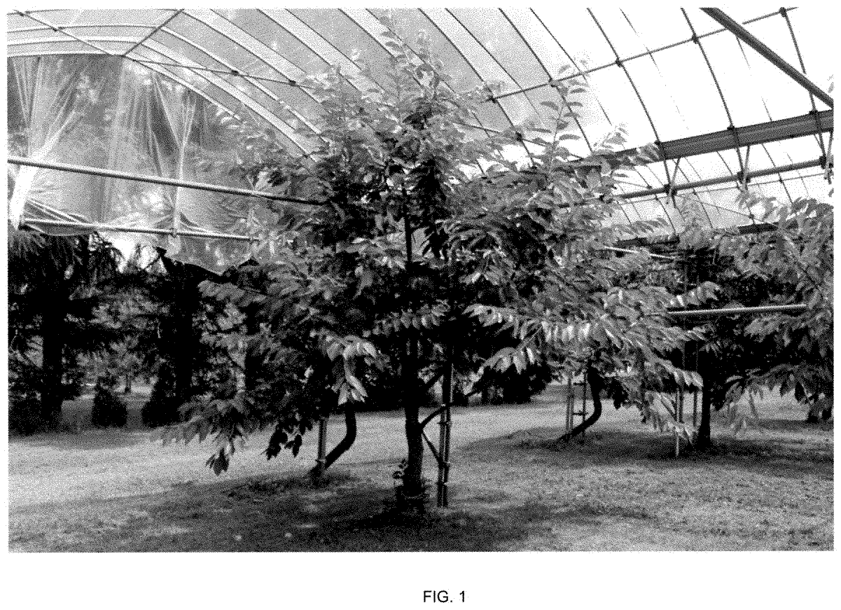

FIG. 2 illustrates flowers of ‘Himari’

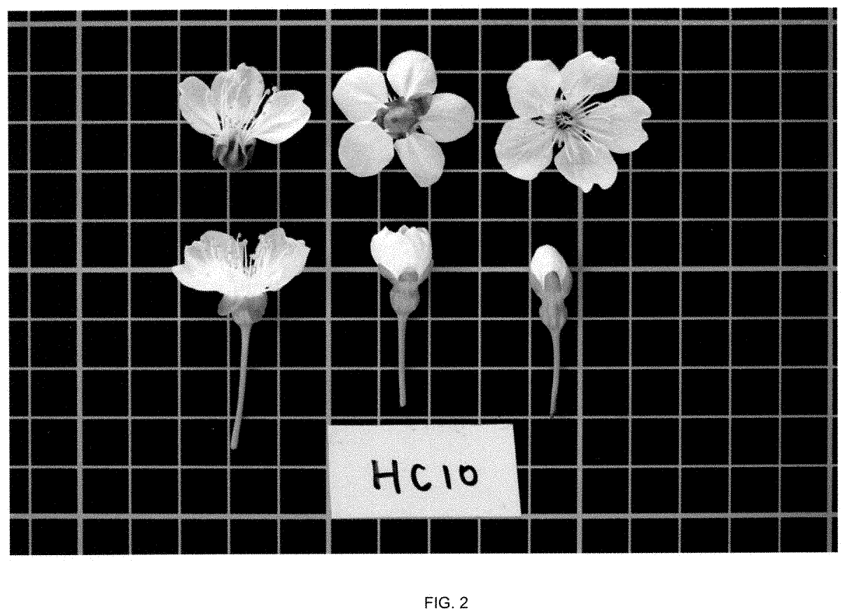

FIG. 3 illustrates parts of ‘Himari’ flowers.

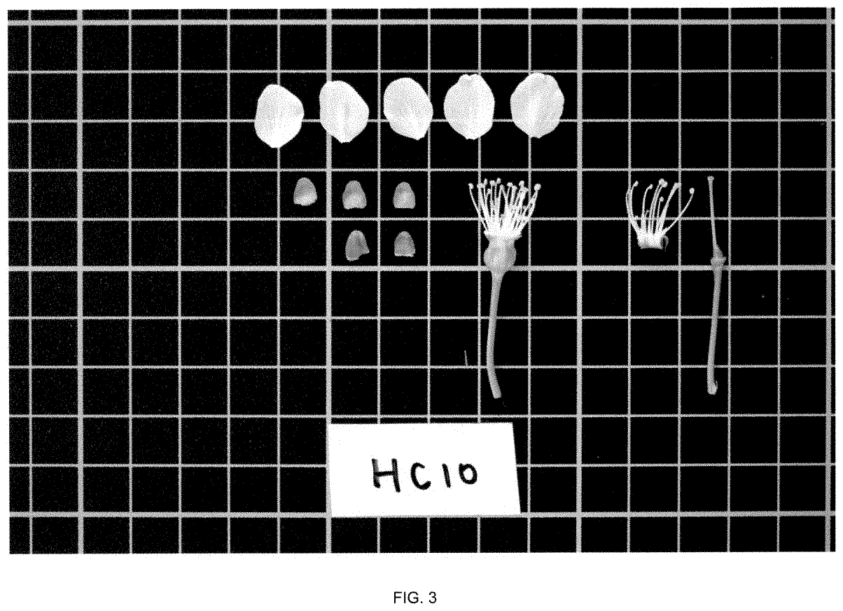

FIG. 4 illustrates ‘Himari’ fruit.

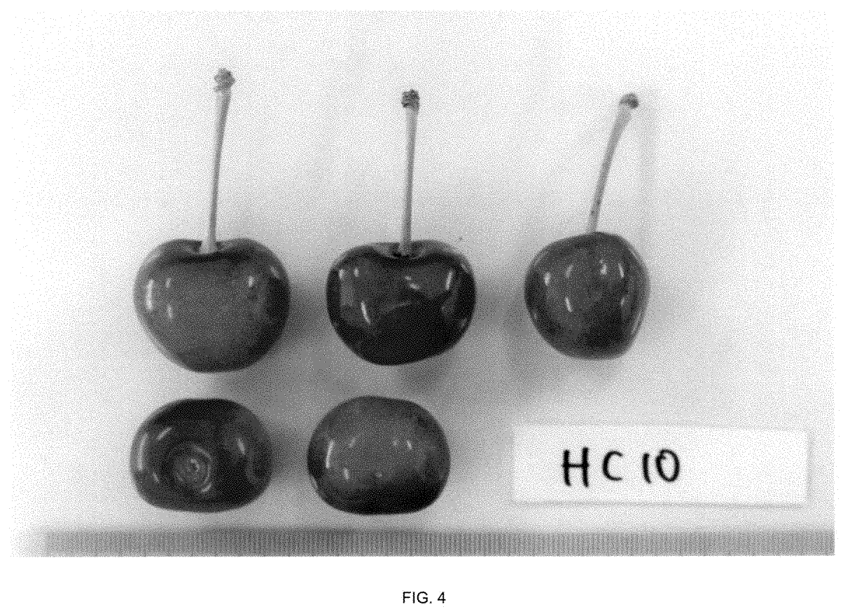

FIG. 5 depicts fruit of comparison variety ‘Sato Nishiki’.

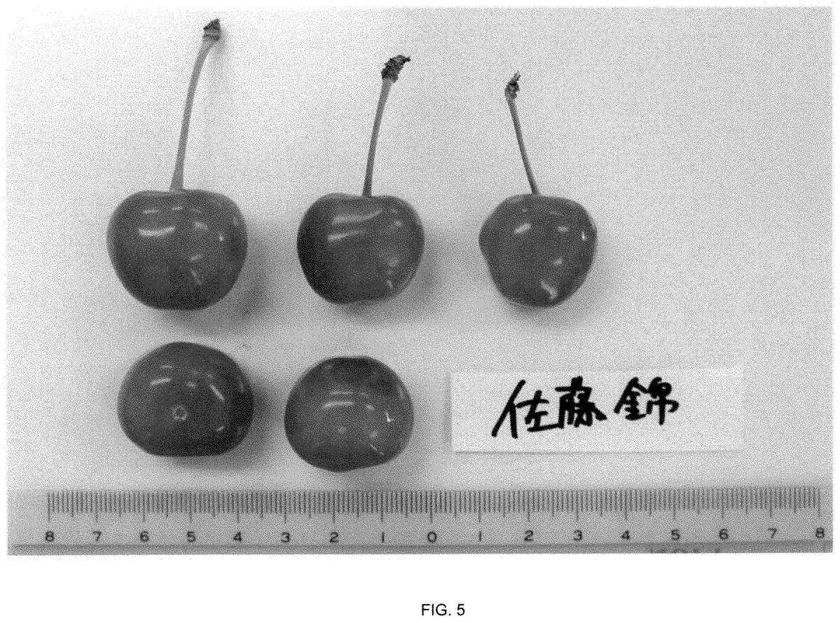

FIG. 6 depicts fruit of comparison variety ‘Nanyo’.

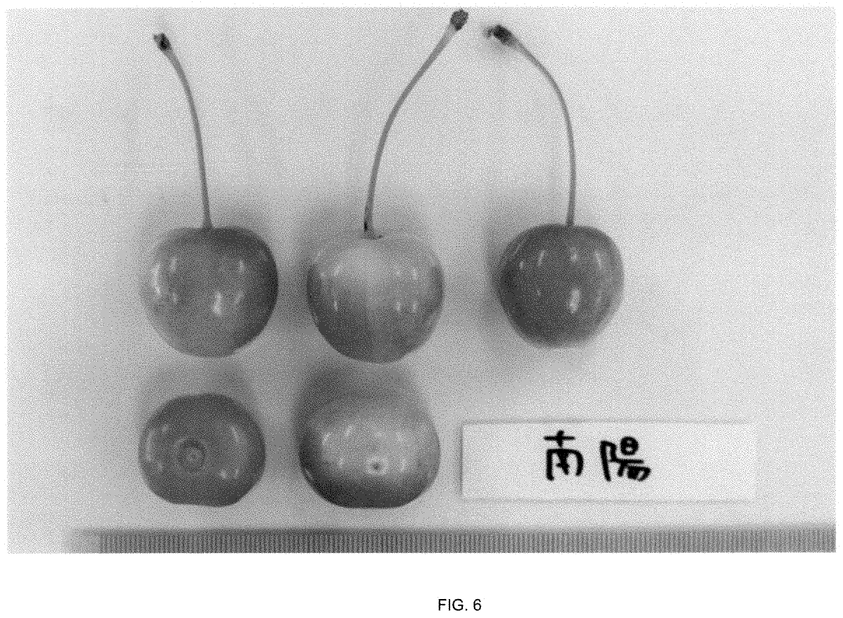

FIG. 7 depicts ‘Himari’ fruit in cross-section.

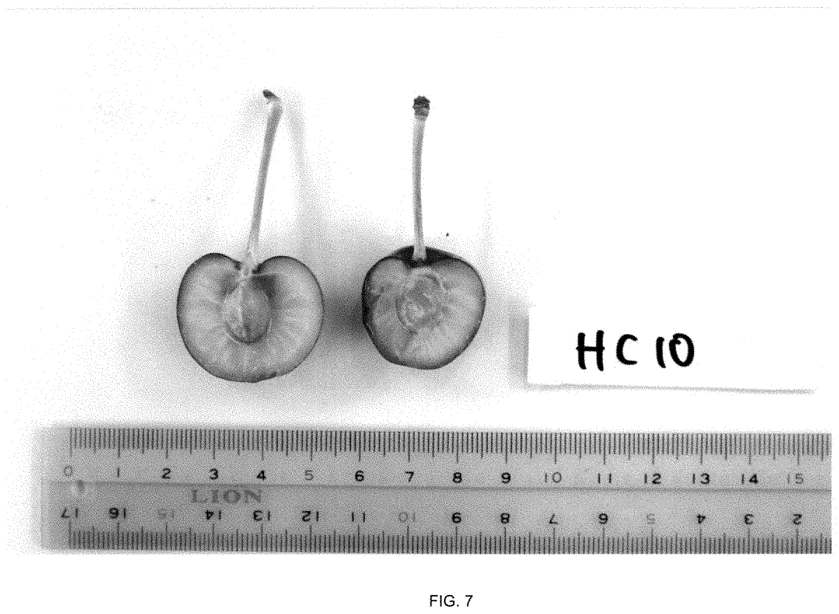

### DETAILED DESCRIPTION

The following is a description of ‘Himari’ trees and fruit that were grown in Hokkaido, Japan. For purposes of this description, ‘Himari’ is referred to by its earlier designation ‘HC10’. The data presented in Table 1 is based on the Characteristic Table for the Japanese Plant Vairety Protection application corresponding to UPOV guidelines. Data from additional trials is also provided in this section.

### Morphological Characteristics

The tree vigor is “medium to strong”, while “strong” for ‘Sato Nishiki’ and “medium to strong” for ‘Nanyo’. The tree habit is “spreading”, while “semi-upright” for ‘Sato Nishiki’ and ‘Nanyo’. Tree branching is “weak to medium”, while “medium” for ‘Sato Nishiki’ and ‘Nanyo’. The bouquet spur is “medium”, which is weaker than “strong” for ‘Sato Nishiki’ and “medium to strong” for ‘Benishuho’ and stronger than “weak” for ‘Nanyo’.

### Ecological Characteristics

The beginning of blooming is “very late”, which is slightly later than “late” for ‘Sato Nishiki’ and equivalent to ‘Nanyo’. The beginning of fruit ripening is “late”, which is later than “medium” for ‘Sato Nishiki’ and equivalent to ‘Nanyo’.

### Fruit Characteristics

The fruit size is “very large”, while “medium” for ‘Sato Nishiki’ and “very large” for ‘Nanyo’. The fruit shape is “reniform”, while “elliptic” for ‘Nanyo’ and “reniform” for ‘Sato Nishiki’ and ‘Benishuho’. The fruit pistil end is “depressed”, while “flat” for ‘Nanyo’ and “depressed” for ‘Sato Nishiki’. The skin color is “dark red”, which is darker than “light red” for ‘Nanyo’ and “red” for ‘Sato Nishiki’. The skin coloration is “medium to strong”, which is slightly stronger than “weak to medium” for ‘Nanyo’ and equivalent to ‘Sato Nishiki’. The skin thickness is “thick”, which is thicker than “medium” for ‘Sato Nishiki’ and “medium to thick” for ‘Nanyo’ and equivalent to ‘Benishuho’. The fruit firmness is “medium to firm”, which is firmer than “medium” for ‘Sato Nishiki’ and “soft to medium” for ‘Nanyo’ and softer than “firm” for ‘Benishuho’. The fruit acidity is “low”, which is equivalent to ‘Sato Nishiki’, ‘Nanyo’, and ‘Benishuho’. The fruit sweetness is “medium to high”, which is higher than “medium” for ‘Sato Nishiki’ and ‘Nanyo’ and lower than “high” for ‘Benishuho’. The fruit juiciness is “strong”, which is equivalent to ‘Sato Nishiki’ and ‘Nanyo’. The stone shape is “small”, which is smaller than ‘Sato Nishiki’ and ‘Nanyo’. The ratio of weight of fruit to weight of stone is “large”, while “small” for ‘Sato Nishiki’ and “medium” for ‘Nanyo’.

### Illustrative Test Results in Comparison Testing

Table 1 provides comparative results from analysis of characteristics of ‘HC10’ compared to ‘Nanyo’, ‘Benishuho’ and ‘Sato Nishiki’ performed from 2019 to 2021 in Hokkaido, Japan.

TABLE 1
Comparison 2019 to 2021,

| | | | | Sato Nishiki | | Nanyo | | Benishuho | |
|---|---|---|---|---|---|---|---|---|---|
| | | HC10 | | (standard) | | (control) | | (comparison) | |
| No. | Characteristics | State | Grade | State | Grade | State | Grade | State | Grade |
| 1 | Tree vigor | medium | 6 | strong | 7 | strong | 8 | medium to | 6 |
| | | to strong | | | | to | | strong | |
| | | | | | | very | | | |
| | | | | | | strong | | | |
| 2 | Tree habit | spreading | 3 | semi | 2 | semi | 2 | spreading | 3 |
| | | | | upright | | upright | | | |
| 3 | Tree branching | weak to | 4 | medium | 5 | medium | 5 | weak to | 4 |
| | | medium | | | | | | medium | |
| 4 | Young shoot: | weak to | 4 | weak | 3 | weak | 3 | medium | 5 |
| | anthocyanin | medium | | | | | | | |
| | coloration of apex | | | | | | | | |
| 5 | Young shoot: | weak | 3 | medium | 5 | weak | 3 | medium to | 6 |
| | pubescence of apex | | | | | | | strong | |
| 9 | Leaf blade length | medium | 5 | medium | 5 | long | 7 | short to | 4 |
| | | | | | | | | medium | |
| — | Bouquet spurs | medium | — | strong | — | weak | — | medium to | — |
| | | | | | | | | strong | |
| 40 | Time of beginning | very late | 9 | late | 7 | very late | 9 | medium | 5 |
| | of flowering | | | | | | | | |
| 41 | Time of beginning | late | 7 | medium | 5 | late | 7 | medium to | 6 |
| | of fruit ripening | | | | | | | late | |
| 20 | Fruit size | very large | 9 | medium | 5 | very | 9 | medium | 5 |
| | | | | | | large | | | |
| 21 | Fruit shape | reniform | 2 | reniform | 2 | elliptic | 5 | reniform | 2 |
| 22 | Fruit pistil end | depressed | 3 | depressed | 3 | flat | 2 | depressed | 3 |
| 23 | Fruit suture | strongly | 3 | weakly | 2 | strongly | 3 | strongly | 3 |
| | | conspicuous | | conspicuous | | conspicuous | | conspicuous | |
| 27 | Color of skin | dark red | 7 | red | 5 | light red | 4 | red | 5 |
| — | Coloration of skin | medium | — | medium | — | weak to | — | medium | — |
| | | to strong | | to strong | | medium | | | |
| 30 | Thickness of skin | thick | 7 | medium | 5 | medium | 6 | thick | 7 |
| | | | | | | to thick | | | |
| 31 | Color of flesh | cream | 1 | cream | 1 | cream | 1 | yellow | 2 |
| 33 | Fruit firmness | medium | 6 | medium | 5 | soft to | 4 | firm | 7 |
| | | to firm | | | | medium | | | |
| 34 | Fruit acidity | low | 1 | low | 1 | low | 1 | low | 1 |
| 35 | Fruit sweetness | medium | 6 | medium | 5 | medium | 5 | high | 7 |
| | | to high | | | | | | | |
| 36 | Fruit juiciness | strong | 7 | strong | 7 | strong | 7 | medium | 5 |
| 37 | Stone size | small | 3 | medium | 5 | small to | 4 | medium | 5 |
| | | | | | . | medium | | | |
| 38 | Stone shape in | broad | 2 | medium | 1 | medium | 1 | circular | 3 |
| | ventral view | elliptic | | elliptic | | elliptic | | | |
| 39 | Ratio weight of | large | 7 | small | 3 | medium | 5 | small to | 4 |
| | fruit/weight of | | | | | | | medium | |
| | stone | | | | | | | | |

### Test Results in Growing Fields

Test results in growing fields were obtained as follows. Grafting was in 2013 and the planting year was 2014. In the case of grafting, the following year is considered the first year. The rootstock employed was ‘Colt’. Plants were planted at a distance of 5×4 meters. The number of repeat trees was three. Rain cover was present. The tree form was the main trunk form and other cultivation management was in accordance with conventional practices. For grafting, there were five trees for ‘HC10’. The number of trees tested for each year from 2014-2021 is shown in Table 2. There were cases where snow damage, animal damage, and tree vigor decay caused tree death, resulting in a decrease in number of test trees from the time of planting.

TABLE 2
Number of tested trees

| Line/ | Test Year | | | | | | | |
|---|---|---|---|---|---|---|---|---|
| Variety | 2014 | 2015 | 2016 | 2017 | 2018 | 2019 | 2020 | 2021 |
| HC10 | 5 | 5 | 5 | 5 | 5 | 3 | 3 | 3 |
| Sato Nishiki | 3 | 3 | 3 | 3 | 3 | 3 | 3 | 3 |
| Nanyo | 3 | 3 | 3 | 3 | 3 | 3 | 3 | 2 |
| Benishuho | 3 | 3 | 3 | 3 | 3 | 3 | 2 | 2 |

Trunk circumference was measured at a height of 10 cm above the grafting portion. The tree height is the maximum tree height. Tree width is the maximum width of the crown, averaged in row and inter-row directions. The length, thickness and direction of elongation of young shoots were compared. Young shoot length was measured at the tip of offshoot at a height slightly above eye levels (about 5 to 8 shoots).

Growth stages were assessed as follows. Germination stage was observed when three or more leaf buds having loosened scales and initial green were found on offshoots. The blooming stage was considered to begin when continuous blooming began. Full bloom was considered to be when about 80% of blossoms bloomed. More than 80% of bloom shedding was considered to be when petals fall. The harvest stage “prime” was considered to be the day when yield exceeded half.

Productivity was determined as follows. Bouquet spurs observations were based on the density and number of bouquet spurs attached to branches of 2 years or older. The number of floral buds per bouquet spur were assessed on a scale of 1 to 5 with less than or equal to 3 being assessed as “1”, about 5 being assessed as “3” and 7 or more being assess as “5”. The number of fruiter per bouquet spur was the average number of fruit among about 100 spurs at a height slightly above eye level. The yield is the value obtained by multiplying the number of fruits by average fruit weight (kg).

Characteristics considered in evaluating fruit quality included fruit cracking on tree, varying from “0” (None) to “5” (Extreme); Uniformity of fruit size (from “1” (Poor) to “5” (“Good”); average transverse diameter of fruit; fruit size (average weight); skin coloration from “1” (Weak) to “5” (Strong). Skin thickness (Thin to Thick) and fruit firmness (Soft to Firm) was based on sensory analysis. Maximum breaking stress of flesh was measured using a texture analyzer (TA.XT plusC by EKO Instruments Co., Ltd). when a plunger with diameter of 3 mm was pierced at 2 mm/sec on peeled equatorial portion of fruit in transverse diameter direction of fruit core (15 fruits each time). Acidity was assessed in terms of malic acid converted from titration value of 10 or more squeezed mature fruits from “1” (Low) to “5” (High). Sensory evaluations was also performed for acidity, sweetness, and juiciness of mature fruit.

Tree growth and vigor are shown in Table 3 and 4 below. The comparison showed that ‘HC10’ was less than ‘Nanyo’ and equivalent to ‘Sato Nishiki’ in trunk circumference. The tree vigor was “medium to strong”, which was slightly weaker than ‘Nanyo’. The tree habit was “spreading” and the tree branching was “weak to medium”. The young shoot length at tip of offshoot was short. The ratio of young shoots/offshoots was small to medium.

TABLE 3
Tree Growth

| | Line/ | Year (tree age) | | | | | | |
|---|---|---|---|---|---|---|---|---|
| | Variety | 2015 (3) | ′16 (4) | ′17 (5) | ′18 (6) | 19 (7) | ′20 (8) | ′21 (9) |
| Trunk | HC10 | 9.1 | 14.8 | 21.7 | 26.8 | 32.5 | 40.4 | 45.2 |
| circumference | Sato Nishiki | 8.0 | 13.4 | 20.8 | 28.2 | 35.0 | 42.5 | 48.6 |
| (cm) | Nanyo | 8.5 | 12.2 | 18.0 | 25.4 | 30.4 | 46.6 | 53.4 |
| | Benishuho | 10.2 | 16.5 | 22.6 | 29.0 | 32.4 | 37.8 | 42.7 |
| Tree height | HC10 | 2.4 | 3.0 | 3.7 | 3.6 | 4.0 | 4.2 | 4.5 |
| (m) | Sato Nishiki | 2.2 | 3.0 | 3.6 | 4.0 | 4.1 | 4.2 | 4.6 |
| | Nanyo | 1.5 | 2.4 | 2.9 | 3.3 | 3.6 | 4.6 | 5.0 |
| | Benishuho | 2.3 | 2.8 | 3.5 | 3.6 | 3.7 | 4.3 | 4.2 |
| Tree width | HC10 | 0.8 | 1.2 | 2.0 | 2.5 | 3.2 | 3.7 | 4.0 |
| (m) | Sato Nishiki | 0.6 | 1.1 | 1.7 | 2.3 | 2.7 | 3.5 | 4.0 |
| | Nanyo | 0.6 | 0.9 | 1.4 | 1.9 | 2.5 | 3.9 | 4.3 |
| | Benishuho | 0.7 | 1.2 | 1.8 | 2.5 | 2.7 | 3.3 | 4.1 |

The value represents an average of all tested trees (the same applies hereinafter).

TABLE 4
Tree vigor and tree habit

| | Tree Tree vigor (1 weak to 5 strong) | | | | | Tree habit (1 upright to 5 spreading)¹⁾ | | | | | |
|---|---|---|---|---|---|---|---|---|---|---|---|
| | Year | | | ′19 to ′21 | | Year | | | ′19 to ′21 | | |
| Line/Variety | 2019 | 2020 | 2021 | Avg. | Decision | 2019 | 2020 | 2021 | Avg. | Decision | |
| HC10 | 4.7 | 4.0 | 2.7 | 3.8 | medium | 4.0 | 4.0 | 4.3 | 4.1 | spreading | |
| | | | | | to strong | | | | | | |
| Sato Nishiki | 4.7 | 4.7 | 3.7 | 4.3 | strong | 3.0 | 3.0 | 2.7 | 2.9 | semi-upright | |
| Nanyo | 5.0 | 5.0 | 4.5 | 4.8 | very | 3.7 | 3.0 | 3.5 | 3.4 | semi-upright | |
| | | | | | strong | | | | | | |
| Benishuho | 3.5 | 4.5 | 3.0 | 3.7 | medium | 3.5 | 3.5 | 4.5 | 3.8 | spreading | |
| | | | | | to strong | | | | | | |
| | | | | | | Young shoot length at | | | | | |
| | Tree branching (1 weak to 5 strong) | | | | | tip of offshoot (cm) | | | young shoots/offshoots | | |
| | Year | | | ′19 to ′21 | | Year | | ′20 to ′21 | Year | | ′20 to ′21 |
| Line/Variety | 2019 | 2020 | 2021 | Avg. | Decision | 2020 | 2021 | Avg. | 2020 | 2021 | Avg. |
| HC10 | 4.3 | 3.7 | 3.0 | 3.7 | weak to | 48.4 | 51.8 | 50.1 | 3.3 | 3.2 | 3.3 |
| | | | | | medium | | | | | | |
| Sato Nishiki | 5.0 | 5.0 | 4.0 | 4.7 | medium | 53.5 | 55.3 | 54.4 | 4.2 | 4.2 | 4.2 |
| Nanyo | 4.0 | 5.0 | 4.0 | 4.3 | medium | 64.9 | 67.0 | 66.0 | 3.6 | 3.8 | 3.7 |
| Benishuho | 3.5 | 3.0 | 4.0 | 3.5 | weak to | — | — | — | — | — | — |
| | | | | | medium | | | | | | |

Growth stage and S genotype are shown in Tables 5 and 6. The comparison shows that ‘HC10’ was equivalent to ‘Sato Nishiki’ and ‘Nanyo’ in germination stage. The blooming stage and the harvest stage were “very late” and “late”, respectively, equivalent to ‘Nanyo’, but later than ‘Sato Nishiki’. The S genotype of ‘HC10’ is S₁S3, which differs from S3S6 of ‘Sato Nishiki’ and ‘Nanyo’, S1S6 of ‘Hokko’, and S₄S₆ of ‘Benishuho’. ‘HC10’ is thus cross-compatible. In addition, since most of the blooming stages overlap, they function as pollinator trees among each other's cultivars.

TABLE 5
Germination stage, blooming, and harvest stage

| | | Germination | Blooming stage | | | | Harvest stage | | | |
|---|---|---|---|---|---|---|---|---|---|---|
| | | stage | Beginning | | Full | Falling | Beginning | | Prime | End |
| | | (Month/ | (Month/ | | (Month/ | (Month/ | (Month/ | | (Month/ | (Month/ |
| Line/Variety | Year | Day) | Day) | Decision | Day) | Day) | Day) | Decision | Day) | Day) |
| HC10 | 2019 | 4/17 | 5/9 | | 5/14 | 5/23 | 7/15 | | 7/20 | 7/26 |
| | 2020 | 4/4 | 5/12 | | 5/17 | 5/28 | 7/16 | | 7/23 | 7/31 |
| | 2021 | 4/3 | 5/12 | | 5/17 | 6/1 | 7/19 | | 7/26 | 8/5 |
| | Avg. | 4/8 | 5/11 | very late | 5/16 | 5/28 | 7/17 | late | 7/23 | 7/31 |
| Sato Nishiki | 2019 | 4/17 | 5/8 | | 5/13 | 5/21 | 7/1 | | 7/4 | 7/8 |
| | 2020 | 4/2 | 5/11 | | 5/15 | 5/26 | 7/1 | | 7/5 | 7/10 |
| | 2021 | 4/2 | 5/9 | | 5/14 | 5/29 | 7/7 | | 7/10 | 7/15 |
| | Avg. | 4/7 | 5/9 | late | 5/14 | 5/25 | 7/3 | medium | 7/6 | 7/11 |
| Nanyo | 2019 | 4/18 | 5/11 | | 5/15 | 5/22 | 7/15 | | 7/20 | 7/25 |
| | 2020 | 4/2 | 5/12 | | 5/18 | 5/28 | 7/16 | | 7/20 | 7/24 |
| | 2021 | 4/3 | 5/11 | | 5/17 | 5/30 | 7/22 | | 7/26 | 7/30 |
| | Avg. | 4/8 | 5/11 | very late | 5/17 | 5/27 | 7/18 | late | 7/22 | 7/26 |
| Benishuho | 2019 | 4/15 | 5/6 | | 5/11 | 5/21 | 7/8 | | 7/15 | 7/22 |
| | 2020 | 4/2 | 5/8 | | 5/13 | 5/22 | 7/13 | | 7/20 | 7/27 |
| | 2021 | 3/30 | 5/8 | | 5/14 | 5/24 | 7/12 | | 7/19 | 7/26 |
| | Avg. | 4/5 | 5/7 | medium | 5/13 | 5/22 | 7/11 | medium | 7/18 | 7/25 |
| | | | | | | | | to late | | |

Note)
The emphasized portion indicates the standard state of the variety according to the Examination Guidelines for Agricultural, Forest, or Aquatic Plant Species.

TABLE 6
S Genotyping Results (Central Station, Biotechnology Group, 2016)

| | | | | | | | | | | | | | | | | Cultivated |
|---|---|---|---|---|---|---|---|---|---|---|---|---|---|---|---|---|
| | | | | | | | | | | | | | | | | area ratio |
| | S genotype | | | | | | | | | | | | | | Genotype | (%) ¹⁾ |
| Line/Variety | S₁ | S₂ | S₃ | S₄ | S₄′ | S₅ | S₆ | S₇ | S₉ | S₁₀ | S₁₂ | S₁₃ | S₁₄ | S₁₆ | decision | (2018) |
| HC10 | + | | + | | | | | | | | | | | | S₁S₃ | — |
| Sato Nishiki | | | + | | | | + | | | | | | | | S₃S₆ | 46 |
| Nanyo | | | + | | | | + | | | | | | | | S₃S₆ | 16 |
| Hokko | + | | | | | | + | | | | | | | | S₁S₆ | 25 |
| Benishuho | | | | + | | | + | | | | | | | | S₄S₆ | 7 |

Ratio of Cultivation Area in Hokkaido according to the 2018 Production Trends of Specialty Fruit Trees Research (Ministry of Agriculture, Forestry and Fisheries).

Blooming and yield results are shown in Tables 7 and 8. The density of bouquet spurs of ‘HC10’ was “medium”, which is more than ‘Nanyo’ and lower than ‘Sato Nishiki’. ‘HC10’ was equivalent to ‘Nanyo’ in floral buds per bouquet spur. The number of fruits per bouquet spur was larger than that of ‘Nanyo’ and less than that of ‘Sato Nishiki’. The fruiting age was 5 or 6 years, equivalent to ‘Sato Nishiki’ and ‘Nanyo’. ‘HC10’ was larger than ‘Nanyo’ and less than ‘Sato Nishiki’ in the number of fruits per tree. ‘HC10’ was larger than ‘Nanyo’ and less than ‘Sato Nishiki’ in yield.

TABLE 7
Bouquet spurs, floral buds, and fruit-setting property

| | | | | | | Floral buds per | Number of |
|---|---|---|---|---|---|---|---|
| | Bouquet spurs (1 weak to 5 strong) | | | | | bouquet spur¹⁾ | fruits per |
| | Year | | | ′19 to ′21 | | (1 weak to 5 | bouquet spur¹⁾ |
| Line/Variety | 2019 | 2020 | 2021 | Avg. | Decision | strong) | (pieces) |
| HC10 | 3.0 | 2.7 | 2.3 | 2.7 | medium | 1.0 | 1.4 |
| Sato Nishiki | 4.7 | 5.0 | 4.0 | 4.6 | strong | 2.7 | 2.4 |
| Nanyo | 1.0 | 2.3 | 2.0 | 1.8 | weak | 1.0 | 0.6 |
| Benishuho | 3.5 | 4.0 | 4.0 | 3.8 | medium to | 2.5 | 3.0 |
| | | | | | strong | | |

¹⁾Researched only in 2021

TABLE 8
Number of fruits and yield per tree

| | Number of fruits (/tree) | | | | | | |
|---|---|---|---|---|---|---|---|
| | Test Year (tree age) | | | | | | |
| Line/Variety | 2015 (3) | ′16 (4) | ′17 (5) | ′18 (6) | ′19 (7) | ′20 (8) | ′21 (9) |
| HC10 | 1 | 0 | 1 | 1 | 224 | 427 | 303 |
| Sato Nishiki | 0 | 0 | 0 | 29 | 577 | 563 | 1331 |
| Nanyo- | 0 | 0 | 0 | 3 | 83 | 91 | 342 |
| Benishuho | 0 | 0 | 1 | 43 | 1299 | 814 | 1288 |

| | | | | | Cumulative | Cumulative yield |
|---|---|---|---|---|---|---|
| | Yied (kg/tree)¹⁾ | | | | yield relative | relative to |
| | Test Year (tree age) | | | Cumulative | to control | standard |
| Line/Variety | ′19 (7) | ′20 (8) | ′21 (9) | yield | (%) | (%) |
| HC10 | 2.1 | 4.7 | 3.5 | 10.4 | 198 | 54 |
| Sato Nishiki | 4.0 | 4.3 | 11.0 | 19.2 | 366 | (100) |
| Nanyo | 0.9 | 0.9 | 3.5 | 5.3 | (100) | 27 |
| ,Benishuho | 9.0 | 7.0 | 11.2 | 27.2 | 518 | 142 |

¹⁾Yield was calculated by fruit number × fruit size.

In fruit quality observations determined at prime of harvest (see, Table 9), the fruit transverse diameter of ‘HC10’ was 28.6 mm, which was larger than those of ‘Sato Nishiki’, ‘Nanyo’, and ‘Benishuho’. The fruit size was 10.7 g and “very large”, equivalent to ‘Nanyo’, indicating a large size line. The skin coloration was “medium to strong”, which was obviously stronger than ‘Nanyo’ and was equivalent to ‘Sato Nishiki’. The skin thickness was “thick”, which was thicker than ‘Sato Nishiki’ and ‘Nanyo’. The fruit firmness was “medium to firm”, which was firmer than ‘Sato Nishiki’ and ‘Nanyo’ and softer than ‘Benishuho’. The acidity was higher than those of ‘Nanyo’ and ‘Benishuho’ and equivalent to ‘Sato Nishiki’. The sugar content was 21.5° Brix, which was higher than those of ‘Sato Nishiki’ and ‘Nanyo’ and slightly lower than that of ‘Benishuho’. In the sensory analysis, the fruit acidity was “low”, equivalent to ‘Sato Nishiki’ and ‘Nanyo’. The sweetness was “medium to high”, which was higher than those of ‘Sato Nishiki’ and ‘Nanyo’ and slightly lower than ‘Benishuho’. The fruit juiciness was “strong”, equivalent to ‘Sato Nishiki’ and ‘Nanyo’. The stone size was “small”, which was smaller than those of ‘Sato Nishiki’ and ‘Nanyo’. The ratio of fruit size to the stone was “large”.

TABLE 9
Fruit quality at prime of harvest

| | | | | | | | | | | | Maximum |
|---|---|---|---|---|---|---|---|---|---|---|---|
| | | Fruit | | | Skin coloration | | Skin thickness | | Fruit firmness | | breaking |
| | | transverse | | | (1 weak | | (1 thin | | (1 soft | | stress |
| Line/ | | diameter | Fruit size | | to 5 | | to 5 | | to 5 | | of flesh¹⁾ (g) |
| Variety | Year | (mm) | (g) | Decision | strong) | Decision | thick) | Decision | firm) | Decision | (g) |
| HC10 | 2019 | 29.0 | 9.7 | | 3.8 | | 5.0 | | 3.2 | | — |
| | 2020 | 27.9 | 11.0 | | 3.8 | | 5.0 | | 3.7 | | — |
| | 2021 | 29.0 | 11.5 | | 3.3 | | 5.0 | | 2.7 | | 74.3 |
| | Avg. | 28.6 | 10.7 | very | 3.6 | medium | 5.0 | thick | 3.2 | medium to | — |
| | | | | large | | to strong | | | | firm | |
| Sato | 2019 | 25.6 | 7.0 | | 4.0 | | 2.7 | | 1.7 | | — |
| Nishiki | 2020 | 24.0 | 7.6 | | 4.0 | | 4.7 | | 2.3 | | — |
| | 2021 | 24.9 | 8.2 | | 3.7 | | 3.0 | | 2.0 | | 70.2 |
| | Avg. | 24.8 | 7.6 | medium | 3.9 | medium | 3.4 | medium | 2.0 | medium | — |
| | | | | | | to strong | | | | | |
| Nanyo | 2019 | 27.6 | 10.0 | | 2.5 | | 3.5 | | 2.0 | | — |
| | 2020 | 25.4 | 9.1 | | 2.0 | | 5.0 | | 1.0 | | — |
| | 2021 | 26.0 | 9.6 | | 2.5 | | 4.0 | | 2.0 | | 52.9 |
| | Avg. | 26.3 | 9.6 | very | 2.3 | weak to | 4.2 | medium | 1.7 | soft to | — |
| | | | | large | | medium | | to thick | | medium | |
| Benishuho | 2019 | 24.8 | 6.7 | | 3.3 | | 4.8 | | 4.0 | | — |
| | 2020 | 25.5 | 8.4 | | 4.0 | | 5.0 | | 5.0 | | — |
| | 2021 | 26.4 | 8.8 | | 2.5 | | 4.5 | | 3.5 | | 95.1 |
| | Avg. | 25.6 | 7.9 | medium | 3.3 | medium | 4.8 | thick | 4.2 | firm | — |

| | | | | | Fruit acidity | | Fruit sweetness | | Fruit juiciness | | | | Ratio weight | |
|---|---|---|---|---|---|---|---|---|---|---|---|---|---|---|
| | | Acidity | Sugar | Sugar | (1 low | | (1 low | | (1 weak | | Stone size³⁾ | | of fruit/weight | |
| Line/ | | (g/100 | content | acid | to 5 | Deci- | to 5 | Deci- | to 5 | Deci- | | Deci- | of stone | |
| Variety | Year | mL) | (° Brix) | ratio | high) | sion | high) | sion | strong) | sion | (g) | sion | | Decision |
| HC10 | 2019 | 0.61 | 20.1 | 33.0 | 1.8 | | 3.3 | | 3.8 | | — | | — | |
| | 2020 | 0.58 | 21.9 | 37.8 | 2.3 | | 3.8 | | 4.0 | | 0.33 | | 33.3 | |
| | 2021 | 0.60 | 22.5 | 37.5 | 2.0 | | 3.7 | | 5.0 | | 0.31 | | 37.5 | |
| | Avg. | 0.60 | 21.5 | 36.0 | 2.1 | low | 3.6 | medium | 4.3 | strong | 0.32 | small | 35.4 | large |
| | | | | | | | | to high | | | | | | |
| Sato | 2019 | 0.54 | 19.1 | 35.4 | 2.0 | | 3.3 | | 3.3 | | — | | — | |
| Nishiki | 2020 | 0.56 | 17.6 | 31.4 | 1.7 | | 3.0 | | 4.7 | | 0.41 | | 18.5 | |
| | 2021 | 0.58 | 18.6 | 32.1 | 2.0 | | 3.3 | | 4.7 | | 0.37 | | 22.4 | |
| | Avg. | 0.56 | 18.4 | 32.9 | 1.9 | low | 3.2 | medium | 4.2 | strong | 0.39 | medium | 20.5 | small |
| Nanyo | 2019 | 0.53 | 18.0 | 34.0 | 1.0 | | 3.0 | | 3.8 | | — | | — | |
| | 2020 | 0.46 | 21.6 | 47.0 | 1.0 | | 4.0 | | 5.0 | | 0.38 | | 23.9 | |
| | 2021 | 0.53 | 20.0 | 37.7 | 3.0 | | 2.0 | | 4.5 | | 0.33 | | 29.4 | |
| | Avg. | 0.51 | 19.9 | 39.2 | 1.7 | low | 3.0 | medium | 4.4 | strong | 0.35 | small to | 26.7 | medium |
| | | | | | | | | | | | | medium | | |
| Benishuho | 2019 | 0.49 | 20.8 | 42.4 | 1.4 | | 3.6 | | 2.8 | | — | | — | |
| | 2020 | 0.49 | 24.5 | 50.0 | 1.3 | | 5.0 | | 3.0 | | 0.34 | | 24.7 | |
| | 2021 | 0.40 | 21.2 | 53.0 | 1.0 | | 3.0 | | 3.3 | | 0.39 | | 22.6 | |
| | Avg. | 0.46 | 22.2 | 48.2 | 1.2 | low | 3.9 | high | 3.0 | medium | 0.37 | medium | 23.6 | small to |
| | | | | | | | | | | | | | | medium |

¹⁾measured only in 2021.

Incidence of cold damage, frost damage, and disease and pestilence were also evaluated. Research criteria for cold damage and frost damage are as follows in Table 10. Results are shown in Table 11. Incidence of damage due to Monilinia kusanoi, Monilinia fructicola, Rhacochlaena japonica, and Drosophila suzukii was also assessed. The incidence of cold damage of ‘HC10’ was more than that of ‘Hokko’ and equivalent to those of ‘Sato Nishiki’, ‘Nanyo’, and ‘Benishuho’. The incidence of frost damage was equivalent to those of ‘Sato Nishiki’, ‘Nanyo’, and ‘Benishuho’. There was no incidence of disease and pest infestation. No damage due to Monilinia kusanoi, Monilinia fructicola, Rhacochlaena japonica, or Drosophila suzukii was observed, or was damage due to other diseases or pests observed.

TABLE 10

| | | | |
|---|---|---|---|
| Items | Research Criteria | Research | Unit |

The degree of cold damage during the
winter period is evaluated by observation.

| | | | |
|---|---|---|---|
| Degree | 0: | none | obser- |
| of cold | 1: | death of part of floral buds | vation |
| damage | | (without influence on yield) | |
| | 2: | death of many floral buds | |
| | | (with influence on yield) | |
| | 3: | death of leaf buds (spur leaf buds) | |
| | 4: | death of branches | |
| | | (vegetative branch leaf buds) | |
| | 5: | death | |

The degree of frost damage in early spring
is evaluated based on the degree of pistil loss.

| | | | | | | | |
|---|---|---|---|---|---|---|---|
| Degree | 0 | 1 | 2 | 3 | 4 | 5 | obser- |
| of frost | 0% | ~20% | 20 to | 40 to | 60 to | 80%~ | vation |
| damage | | | 40% | 60% | 80% | | |

TABLE 11
Incidence of cold damage and frost damage

| | Incidence of cold damage | | | Incidence of frost damage | | |
|---|---|---|---|---|---|---|
| | (0 None to 5 Extreme) | | | (0 None to 5 Extreme) | | |
| Line/ | Year | | | Year | | |
| Variety | 2019 | 2020 | 2021 | 2019 | 2020 | 2021 |
| HC10 | 0.0 | 0.0 | 1.3 | 0.0 | 0.0 | 1.0 |
| Sato | 0.0 | 0.0 | 1.0 | 0.0 | 0.0 | 1.0 |
| Nishiki | | | | | | |
| Nanyo | 0.0 | 0.0 | 1.5 | 0.0 | 0.0 | 1.0 |
| Benishuho | 0.0 | 0.0 | 1.5 | 0.0 | 0.0 | 1.0 |
| Hokko¹⁾ | 0.0 | 0.0 | 0.0 | 0.0 | 0.0 | 1.0 |

¹⁾used only in this research as a standard, variety having a cold hardiness of “strong”

### Additional Trial Results

Additional test results are presented below of test locations Fukagawa City, Mashike town, and Yoichi Town. The planting scheme and number of tested trees are shown in Table 12.

TABLE 12
Planting

| | | Settled | | | Number | |
|---|---|---|---|---|---|---|
| Test | Grafting | planting | | Planting | of repeat | Rain |
| location | year | year | Rootstock | Distance | trees | cover |
| Fukagawa | 2013 | 2014 | Colt | 6 × 4 m | 3 | present |
| City | | | | | | |
| Mashike | 2014 | 2015 | Colt | 7 × 3.6 m | 3 | present |
| Town | | | | | | |
| Yoichi | 2013 | 2014 | Colt | 7.2 × 3.6 m | 3 | present |
| Town | | | | | | |

Number of tested trees

| Test | Line/ | Test Year | | | | | | | |
|---|---|---|---|---|---|---|---|---|---|
| location | Variety | 2014 | 2015 | 2016 | 2017 | 2018 | 2019 | 2020 | 2021 |
| Fukagawa | HC10 | 3 | 3 | 3 | 3 | 3 | 3 | 3 | 3 |
| City | Sato | 3 | 2 | 2 | 2 | 2 | 2 | 2 | 2 |
| | Nishiki | | | | | | | | |
| | Nanyo | 3 | 2 | 2 | 2 | 2 | 2 | 2 | 2 |
| | Benishuho | 3 | 3 | 3 | 3 | 3 | 3 | 3 | 3 |
| Mashike | HC10 | | 3 | 3 | 3 | 3 | 3 | 3 | 3 |
| Town | Sato | | 3 | 3 | 3 | 3 | 3 | 3 | 3 |
| | Nishiki | | | | | | | | |
| Yoichi | HC10 | 3 | 3 | 3 | 3 | 3 | 3 | 3 | 3 |
| Town | Benishuho | 3 | 3 | 3 | 3 | 3 | 3 | 3 | 3 |
| | Sato | 3 | 2 | 2 | 2 | 1 | 1 | 1 | 1 |
| | Nishiki¹⁾ | | | | | | | | |

There were cases where snow damage, animal damage, and tree vigor decay caused tree death, resulting in a decrease in number of test trees from the time of planting.
¹⁾excluded from the results and treated as reference data because of reduction of test trees due to snow damage and animal damage in addition to development of Agrobacterium tumefaciens in remaining trees.

The same criteria were employed for these evaluations as described above. The results (Tables 13 and 14) showed that ‘HC10’ was larger than ‘Sato Nishiki’ and ‘Nanyo’ and equivalent to or slightly larger than ‘Benishuho’ in trunk circumference. The tree vigor was slightly stronger than that of ‘Sato Nishiki’ and equivalent to that of ‘Nanyo’.

TABLE 13
Tree growth

| | Test | | Year (tree age)¹⁾ | | | | | | |
|---|---|---|---|---|---|---|---|---|---|
| Items | location | Line/Variety | 2015 (3) | ′16 (4) | ′17 (5) | ′18 (6) | ′19 (7) | ′20 (8) | ′21 (9) |
| Trunk | Fukagawa | HC10 | 9.3 | 15.2 | 22.2 | 33.0 | 38.0 | 42.9 | 47.8 |
| circumference | City | Sato Nishiki | 7.0 | 12.6 | 18.0 | 24.6 | 29.3 | 32.0 | 34.7 |
| (cm) | | Nanyo | 9.2 | 13.3 | 18.0 | 25.1 | 29.0 | 32.5 | 36.2 |
| | | Benishuho | 10.8 | 17.9 | 24.0 | 32.8 | 36.9 | 40.5 | 45.1 |
| | Mashike | HC10 | 5.8 | 8.0 | 13.8 | 20.1 | 26.3 | 31.7 | 37.2 |
| | Town | Sato Nishiki | 4.1 | 5.4 | 9.0 | 12.8 | 16.4 | 20.0 | 26.0 |
| | Yoichi | HC10 | 9.2 | 16.0 | 22.8 | 32.3 | 35.9 | 42.0 | 49.2 |
| | Town | Benishuho | 10.5 | 17.4 | 23.6 | 30.0 | 33.1 | 38.6 | 43.3 |
| | | (Sato Nishiki) | 7.9 | 13.3 | 15.4 | 23.3 | 27.0 | 31.0 | 37.3 |
| Tree | Fukagawa | HC10 | 2.1 | 3.0 | 3.6 | 4.0 | 3.8 | 4.2 | 4.2 |
| height (m) | City | Sato Nishiki | 1.2 | 2.5 | 3.0 | 3.4 | 3.4 | 3.7 | 3.8 |
| | | Nanyo | 1.7 | 3.1 | 3.6 | 4.2 | 3.8 | 3.8 | 4.4 |
| | | Benishuho | 2.5 | 3.4 | 3.7 | 4.1 | 3.7 | 4.0 | 4.5 |
| | Mashike | HC10 | 1.8 | 2.2 | 3.0 | 4.0 | 4.2 | 4.4 | 4.5 |
| | Town | Sato Nishiki | 1.2 | 1.5 | 2.4 | 3.0 | 3.3 | 3.7 | 3.7 |
| | Yoichi | HC10 | .2.1 | 3.4 | 4.3 | 5.1 | 4.4 | 4.2 | 4.2 |
| | Town | Benishuho | 2.1 | 3.0 | 4.1 | 4.7 | 3.9 | 4.2 | 3.7 |
| | | (Sato Nishiki) | 1.2 | 2.3 | 3.7 | 4.5 | 4.2 | 4.0 | 3.4 |
| Tree width | Fukagawa | HC10 | 1.2 | 1.6 | 2.6 | 3.5 | 3.9 | 4.6 | 5.1 |
| (m) | City | Sato Nishiki | 0.6 | 1.0 | 1.6 | 2.3 | 2.5 | 2.6 | 3.2 |
| | | Nanyo | 1.2 | 1.1 | 2.2 | 2.6 | 2.8 | 3.5 | 4.2 |
| | | Benishuho | 0.6 | 1.7 | 2.6 | 3.4 | 3.8 | 4.1 | 5.0 |
| | Mashike | HC10 | 0.2 | 0.7 | 1.4 | 2.1 | 2.6 | 3.3 | 4.2 |
| | Town | Sato Nishiki | 0.2 | 0.4 | 0.9 | 1.1 | 1.3 | 1.8 | 2.2 |
| | Yoichi | HC10 | 0.8 | 1.7 | 2.7 | 3.1 | 3.8 | 4.6 | 5.1 |
| | Town | Benishuho | 0.8 | 1.5 | 2.3 | 2.8 | 3.8 | 4.5 | 5.0 |
| | | (Sato Nishiki) | 0.3 | 0.9 | 1.4 | 2.1 | 2.1 | 2.6 | 3.1 |

¹⁾2- to 8-year trees only in Mashike town

TABLE 14
Tree Vigor

| | | Tree vigor (1 weak to 5 strong) | | | |
|---|---|---|---|---|---|
| Test | Line/ | Year | | | ′19 to ′21 |
| location | Variety | 2019 | 2020 | 2021 | Avg. |
| Fukagawa | HC10 | 5.0 | 4.7 | 4.7 | 4.8 |
| City | Sato Nishiki | 4.5 | 4.5 | 3.7 | 4.2 |
| | Nanyo | 5.0 | 5.0 | 5.0 | 5.0 |
| | Benishuho | 4.7 | 4.3 | 3.7 | 4.2 |
| Mashike | HC10 | 4.3 | 3.0 | 3.3 | 3.6 |
| Town | Sato Nishiki | 2.3 | 2.3 | 1.3 | 2.0 |
| Yoichi | HC10 | 4.0 | 3.3 | 3.3 | 3.6 |
| Town | Benishuho | 3.0 | 2.3 | 3.0 | 2.8 |
| | (Sato Nishiki) | 4.0 | 2.0 | 2.0 | 2.7 |

Growth stage comparison results are shown in Tables 15-17. The results showed that ‘HC10’ was slightly later than ‘Benishuho’ in germination stage and equivalent to ‘Sato Nishiki’ and ‘Nanyo’. The blooming stage was equivalent to those of ‘Sato Nishiki’ and ‘Nanyo’. Since most of the blooming stages overlap, there is no problem for pollen exchange. The beginning of fruit ripening was 15 to 17-day later than “medium” of ‘Sato Nishiki’ and equivalent to or slightly later than ‘Nanyo’

TABLE 15
Germination stage, Blooming stage, and Harvest stage (Fukagawa City)

| | | Germination | Blooming stage | | | Harvest stage | | |
|---|---|---|---|---|---|---|---|---|
| | | stage | Beginning | Full | Falling | Beginning | Prime | End |
| | | (Month/ | (Month/ | (Month/ | (Month/ | (Month/ | (Month/ | (Month/ |
| Line/Variety | Year | Day) | Day) | Day) | Day) | Day) | Day) | Day) |
| HC10 | 2019 | 4/14 | 5/8 | 5/16 | 5/23 | 7/17 | 7/22 | 7/25 |
| | 2020 | 4/9 | 5/10 | 5/13 | 5/27 | 7/31 | 8/2 | 8/4 |
| | 2021 | 4/12 | 5/12 | 5/16 | 5/26 | 7/25 | 7/25 | 8/4 |
| | Avg. | 4/12 | 5/10 | 5/15 | 5/25 | 7/24 | 7/27 | 8/1 |
| Sato Nishiki | 2019 | 4/14 | 5/7 | 5/14 | 5/22 | 6/26 | 7/3 | 7/11 |
| | 2020 | 4/7 | 5/10 | 5/13 | 5/25 | 7/12 | 7/15 | 7/19 |
| | 2021 | 4/16 | 5/11 | 5/15 | 5/23 | 7/14 | 7/17 | 7/20 |
| | Avg. | 4/12 | 5/9 | 5/14 | 5/23 | 7/7 | 7/12 | 7/17 |
| Nanyo | 2019 | 4/14 | 5/8 | 5/16 | 5/23 | 7/14 | 7/20 | 7/27 |
| | 2020 | 4/9 | 5/11 | 5/13 | 5/27 | 7/23 | 7/27 | 8/1 |
| | 2021 | 4/14 | 5/12 | 5/17 | 5/26 | 7/21 | 7/25 | 7/28 |
| | Avg. | 4/12 | 5/10 | 5/15 | 5/25 | 7/19 | 7/24 | 7/29 |
| Benishuho | 2019 | 4/14 | 5/5 | 5/12 | 5/22 | 7/7 | 7/13 | 7/20 |
| | 2020 | 4/7 | 5/9 | 5/11 | 5/23 | 7/15 | 7/23 | 8/1 |
| | 2021 | 4/12 | 5/8 | 5/11 | 5/21 | 7/16 | 7/21 | 7/27 |
| | Avg. | 4/11 | 5/7 | 5/11 | 5/22 | 7/13 | 7/19 | 7/26 |

TABLE 16
Germination stage, Blooming stage, and Harvest stage (Mashike Town)

| | | Germination | Blooming stage | | | Harvest stage | | |
|---|---|---|---|---|---|---|---|---|
| | | stage | Beginning | Full | Falling | Beginning | Prime | End |
| | | (Month/ | (Month/ | (Month/ | (Month/ | (Month/ | (Month/ | (Month/ |
| Line/Variety | Year | Day) | Day) | Day) | Day) | Day) | Day) | Day) |
| HC10 | 2019 | 4/11 | 5/6 | 5/10 | 5/22 | 7/11 | 7/21 | 8/1 |
| | 2020 | 4/1 | — | — | — | 7/13 | 7/19 | 7/26 |
| | 2021 | 4/11 | 5/7 | 5/11 | 5/20 | 7/18 | 7/21 | — |
| | Avg. | 4/8 | 5/7 | 5/11 | 5/21 | 7/14 | 7/20 | 7/29 |
| Sato Nishiki | 2019 | 4/10 | 5/6 | 5/11 | 5/24 | 6/23 | 6/26 | 6/29 |
| | 2020 | 4/1 | — | — | — | 6/28 | 7/2 | 7/5 |
| | 2021 | 4/11 | 5/7 | 5/12 | 5/21 | 7/6 | 7/9 | 7/11 |
| | Avg. | 4/7 | 5/7 | 5/12 | 5/23 | 6/29 | 7/2 | 7/5 |

TABLE 17
Germination stage, Blooming stage, and Harvest stage (Yoichi Town)

| | | Germination | Blooming stage | | | Harvest stage | | |
|---|---|---|---|---|---|---|---|---|
| | | stage | Beginning | Full | Falling | Beginning | Prime | End |
| | | (Month/ | (Month/ | (Month/ | (Month/ | (Month/ | (Month/ | (Month/ |
| Line/Variety | Year | Day) | Day) | Day) | Day) | Day) | Day) | Day) |
| HC10 | 2019 | 4/10 | 5/5 | 5/9 | 5/18 | 7/12 | 7/19 | 7/26 |
| | 2020 | 4/15 | 5/6 | 5/13 | 5/25 | 7/17 | 7/22 | 7/27 |
| | 2021 | 4/16 | 5/7 | 5/12 | 5/21 | 7/15 | 7/22 | 7/29 |
| | Avg. | 4/14 | 5/6 | 5/11 | 5/21 | 7/15 | 7/21 | 7/27 |
| Benishuho | 2019 | 4/7 | 5/2 | 5/5 | 5/13 | 7/2 | 7/8 | 7/16 |
| | 2020 | 4/3 | 5/3 | 5/8 | 5/13 | 7/10 | 7/17 | 7/24 |
| | 2021 | 4/7 | 5/1 | 5/7 | 5/16 | 7/9 | 7/15 | 7/22 |
| | Avg. | 4/6 | 5/2 | 5/7 | 5/14 | 7/7 | 7/13 | 7/21 |
| (Sato | 2019 | 4/13 | 5/5 | 5/9 | 5/17 | 6/22 | 6/29 | 7/6 |
| Nishiki) | 2020 | 4/11 | 5/8 | 5/12 | 5/22 | 7/4 | 7/8 | 7/13 |
| | 2021 | 4/14 | 5/6 | 5/11 | 5/21 | 7/2 | 7/8 | 7/15 |
| | Avg. | 4/13 | 5/6 | 5/11 | 5/20 | 6/29 | 7/5 | 7/11 |

Blooming and yield assessments are shown in Tables 18-21. In the density of bouquet spurs, ‘HC10’ was higher than ‘Sato Nishiki’ and ‘Benishuho’ and slightly higher than ‘Nanyo’. In floral buds per bouquet spur, ‘HC10’ was obviously lower than ‘Sato Nishiki’ and ‘Benishuho’ in Fukagawa City and Mashike Town, but equivalent to ‘Benishuho’ in Yoichi Town. The fruiting age of ‘HC10’ was 4 or 5 years, equivalent to ‘Sato Nishiki’ and ‘Nanyo’. The yield was equivalent to or larger than ‘Sato Nishiki’ and obviously larger than ‘Nanyo’.

TABLE 18
Bouquet spurs, floral buds, and fruit-setting property

| | | Bouquet spur | | | | Floral buds | Number of |
|---|---|---|---|---|---|---|---|
| | | (1 weak to | | | | per bouquet | fruits per |
| | | 5 strong) | | | | spur¹⁾ | bouquet |
| Test | Line/ | Year | | | ′19 to ′21 | (1 weak to | spur^{1,2)} |
| location | Variety | 2019 | 2020 | 2021 | Avg. | 5 strong) | (pieces) |
| Fukagawa | HC10 | 4.0 | 2.3 | 2.7 | 3.0 | 1.0 | 0.3 |
| City | Sato Nishiki | 5.0 | 4.5 | 4.5 | 4.7 | 3.0 | 1.2 |
| | Nanyo | 4.0 | 1.5 | 2.0 | 2.5 | 2.0 | 0.1 |
| | Benishuho | 5.0 | 5.0 | 4.7 | 4.9 | 3.3 | 1.5 |
| Mashike | HC10 | 2.0 | 1.3 | 1.7 | 1.7 | 1.3 | 0.2 |
| Town | Sato Nishiki | 4.7 | 4.7 | 5.0 | 4.8 | 4.3 | 3.2 |
| Yoichi Town | HC10 | 4.7 | 3.0 | 4.3 | 4.0 | 2.0 | 4.3 |
| | Benishuho | 5.0 | 5.0 | 4.7 | 4.9 | 2.3 | 5.3 |
| | (Sato | 5.0 | 4.0 | 4.0 | 4.3 | 4.0 | 2.8 |
| | Nishiki) | | | | | | |

¹⁾researched only in 2021.
²⁾partial reduction in the number of fruits due to frost damage in Fukagawa City and Mashike Town

TABLE 19
Number of fruits and yield per tree (Fukagawa City)

| | Number of fruits (/tree) | | | | | | |
|---|---|---|---|---|---|---|---|
| Line/ | Test Year (tree age) | | | | | | |
| Variety | 2015 | ′16 | ′17 | ′18 | ′19 | ′20 | ′21 |
| | (3) | (4) | (5) | (6) | (7) | (8) | (9)¹⁾ |
| HC10 | 0 | 0 | 1 | 11 | 171 | 952 | 225 |
| Sato | 0 | 0 | 1 | 54 | 228 | 848 | 286 |
| Nishiki | | | | | | | |
| Nanyo | 0 | 1 | 0 | 14 | 79 | 410 | 48 |
| Benishuho | 3 | 10 | 43 | 160 | 446 | 1854 | 833 |

| | | | | | Cumula- | Cumula- |
|---|---|---|---|---|---|---|
| | Yield (kg/tree)²⁾ | | | | tive yield | tive yield |
| | | | | ′19 to ′21 | relative | relative |
| | Test Year (tree age) | | | cumu- | to | to |
| Line/ | ′19 | ′20 | ′21 | lative | control | standard |
| Variety | (7) | (8) | (9)¹⁾ | yield | (%) | (%) |
| HC10 | 1.7 | 9.8 | 2.3 | 13.7 | 277 | 177 |
| Sato | 1.3 | 4.8 | 1.7 | 7.8 | 157 | (100) |
| Nishiki | | | | | | |
| Nanyo | 0.6 | 3.9 | 0.4 | 5.0 | (100) | 64 |
| Benishuho | 3.4 | 15.8 | 7.6 | 26.9 | 543 | 346 |

¹⁾reduction in the number of fruits and yield due to frost damage
²⁾The yield was calculated by fruit number × fruit size (the same applies to Tables 19 and 20).

TABLE 20
Number of fruits and yield per tree (Mashike Town)

| | Number of fruits (/tree) | | | | | | |
|---|---|---|---|---|---|---|---|
| | Test Year (tree age) | | | | | | |
| Line/ | 2015 | ′16 | ′17 | ′18 | ′19 | ′20 | ′21 |
| Variety | (2) | (3) | (4) | (5) | (6) | (7) | (8)¹⁾ |
| HC10 | 0 | 0 | 4 | 16 | 159 | 406 | 62 |
| Sato Nishiki | 0 | 0 | 9 | 65 | 187 | 558 | 706 |

| | | | | | Cumula- | Cumula- |
|---|---|---|---|---|---|---|
| | Yield (kg/tree) | | | | tive yield | tive yield |
| | | | | ′19 to ′21 | relative | relative |
| | Test Year (tree age) | | | cumu- | to | to |
| Line/ | ′19 | ′20 | ′21 | lative | control | standard |
| Variety | (6) | (7) | (8)¹⁾ | yield | (%) | (%) |
| HC10 | 1.4 | 3.9 | 0.7 | 6.0 | — | 84 |
| Sato Nishiki | 0.7 | 2.4 | 4.0 | 7.1 | — | (100) |

¹⁾reduction in the number of fruits and yield of ‘HC10’ due to frost damage

TABLE 21
Number of fruits and yield per tree (Yoichi Town)

| | Number of fruits (/tree) | | | | | | |
|---|---|---|---|---|---|---|---|
| | Test Year (tree age) | | | | | | |
| Line/ | 2015 | ′16 | ′17 | ′18 | ′19 | ′20 | ′21 |
| Variety | (3) | (4) | (5) | (6) | (7) | (8) | (9) |
| HC10 | 0 | 2 | 8 | 90 | 367 | 1429 | 1781 |
| Benishuho | 6 | 29 | 159 | 497 | 1352 | 1333 | 1287 |
| (Sato | 0 | 9 | 6 | 15 | 298 | 935 | 785 |
| Nishiki) | | | | | | | |

| | | | | | Cumula- | Cumula- |
|---|---|---|---|---|---|---|
| | Yield (kg/tree) | | | | tive yield | tive yield |
| | | | | ′19 to ′21 | relative | relative |
| | Test Year (tree age) | | | cumu- | to | to |
| Line/ | ′19 | ′20 | ′21 | lative | control | standard |
| Variety | (7) | (8) | (9) | yield | (%) | (%) |
| HC10 | 3.9 | 14.9 | 21.3 | 40.1 | — | — |
| Benishuho | 10.3 | 13.1 | 15.4 | 38.7 | — | — |
| (Sato | 1.7 | 5.4 | 4.8 | 11.9 | — | — |
| Nishiki) | | | | | | |

Analyses of fruit quality in these trials are shown in Tables 2-24. The fruit transverse diameter of ‘HC10’ was 27.5 to 28.1 mm, which was larger than those of ‘Sato Nishiki’, ‘Nanyo’, and ‘Benishuho’. The fruit size was 9.9 to 10.8 g, which was larger than ‘Sato Nishiki’, ‘Nanyo’, and ‘Benishuho’, indicating a large size line. In skin coloration, ‘HC10’ was equivalent to ‘Sato Nishiki’ and obviously stronger than ‘Nanyo’. The fruit firmness was slightly firmer than those of ‘Sato Nishiki’ and ‘Nanyo’. The acidity was equivalent to or higher than that of ‘Sato Nishiki’ and higher than ‘Nanyo’. The sugar content was 20.2 to 21.9° Brix, which was higher than ‘Sato Nishiki’ and ‘Nanyo’ and lower than ‘Benishuho’.

TABLE 22
Fruit quality at prime of harvest (Fukagawa City)

| | | Fruit | | Skin | Fruit | Maximum | | | |
|---|---|---|---|---|---|---|---|---|---|
| | | transverse | Fruit | coloration | firmness | breaking | Acidity | Sugar | Sugar |
| Line/ | Test | diameter | size | (1 weak to | (1 soft to | stress¹⁾ | (g/100 | content | acid |
| Variety | Year | (mm) | (g) | 5 strong) | 5 firm) | (g) | ml) | (° Brix) | ratio |
| HC10 | 2019 | 29.1 | 10.1 | 3.0 | 3.0 | | 0.64 | 21.3 | 33.3 |
| | 2020 | 27.5 | 10.3 | 2.7 | 2.7 | | 0.50 | 18.8 | 37.2 |
| | 2021 | 27.7 | 10.1 | 2.0 | 3.0 | 79.9 | 0.64 | 20.6 | 32.3 |
| | Avg. | 28.1 | 10.1 | 2.6 | 2.9 | | 0.59 | 20.2 | 34.0 |
| Sato | 2019 | 24.0 | 5.9 | 4.0 | 2.0 | | 0.63 | 19.7 | 31.3 |
| Nishiki | 2020 | 22.0 | 5.7 | 2.0 | 2.0 | | 0.52 | 17.3 | 33.5 |
| | 2021 | 21.8 | 5.9 | 2.5 | 3.5 | 95.0 | 0.80 | 19.9 | 25.0 |
| | Avg. | 22.6 | 5.8 | 2.8 | 2.5 | | 0.65 | 19.0 | 29.3 |
| Nanyo | 2019 | 25.6 | 7.8 | 2.0 | 3.0 | | 0.51 | 18.7 | 36.9 |
| | 2020 | 26.7 | 9.6 | 1.5 | 2.0 | | 0.40 | 17.1 | 43.0 |
| | 2021 | 26.0 | 8.8 | 1.0 | 2.3 | 63.7 | 0.53 | 18.9 | 35.7 |
| | Avg. | 26.1 | 8.7 | 1.5 | 2.4 | | 0.48 | 18.2 | 38.1 |
| Benishuho | 2019 | 26.5 | 7.7 | 3.8 | 4.3 | | 0.66 | 26.6 | 40.4 |
| | 2020 | 26.1 | 8.8 | 3.3 | 4.7 | | 0.48 | 21.5 | 44.8 |
| | 2021 | 26.4 | 9.1 | 2.7 | 4.7 | 101.9 | 0.56 | 25.3 | 45.6 |
| | Avg. | 26.3 | 8.5 | 3.3 | 4.6 | | 0.56 | 24.5 | 43.3 |

¹⁾researched for maximum breaking stress only in 2021 (the same applies to Tables 22 and 23).

TABLE 23
Fruit quality at prime of harvest (Mashike Town)

| | | Fruit | | Skin | Fruit | Maximum | | | |
|---|---|---|---|---|---|---|---|---|---|
| | | transverse | Fruit | coloration | firmness | breaking | Acidity | Sugar | Sugar |
| Line/ | Test | diameter | size | (1 weak to | (1 soft to | stress | (g/100 | content | acid |
| Variety | Year | (mm) | (g) | 5 strong) | 5 firm) | (g) | ml) | (° Brix) | ratio |
| HC10 | 2019 | 27.8 | 9.0 | 4.0 | 3.3 | | 0.65 | 22.7 | 35.2 |
| | 2020 | 26.0 | 9.5 | 4.0 | 4.2 | | 0.68 | 21.2 | 31.2 |
| | 2021 | 28.8 | 11.4 | 3.7 | 2.3 | 80.8 | 0.66 | 21.6 | 32.6 |
| | Avg. | 27.5 | 9.9 | 3.9 | 3.3 | | 0.66 | 21.9 | 32.9 |
| Sato | 2019 | 20.0 | 3.7 | 4.0 | 3.3 | | 0.50 | 15.6 | 31.0 |
| Nishiki | 2020 | 19.4 | 4.3 | 2.3 | 2.0 | | 0.42 | 14.6 | 34.8 |
| | 2021 | 21.7 | 5.8 | 3.7 | 2.3 | 69.5 | 0.57 | 18.1 | 31.6 |
| | Avg. | 20.3 | 4.6 | 3.3 | 2.6 | | 0.50 | 16.1 | 32.3 |

TABLE 24
Fruit quality at prime of harvest (Yoichi Town)

| | | Fruit | | Skin | Fruit | Maximum | | | |
|---|---|---|---|---|---|---|---|---|---|
| | | transverse | Fruit | coloration | firmness | breaking | Acidity | Sugar | Sugar |
| Line/ | Test | diameter | size | (1 weak to | (1 soft to | stress | (g/100 | content | acid |
| Variety | Year | (mm) | (g) | 5 strong) | 5 firm) | (g) | ml) | (° Brix) | ratio |
| HC10 | 2019 | 28.6 | 10.5 | 4.5 | 3.2 | | 0.56 | 22.9 | 40.5 |
| | 2020 | 27.1 | 10.4 | 4.2 | 2.2 | | 0.48 | 18.5 | 38.8 |
| | 2021 | 28.7 | 11.4 | 3.7 | 2.7 | 72.6 | 0.50 | 19.5 | 38.8 |
| | Avg. | 28.1 | 10.8 | 4.1 | 2.7 | | 0.51 | 20.3 | 39.4 |
| Benishuho | 2019 | 26.4 | 7.6 | 4.7 | 4.0 | | 0.68 | 23.9 | 35.2 |
| | 2020 | 26.9 | 9.8 | 3.5 | 3.8 | | 0.52 | 23.0 | 44.3 |
| | 2021 | 26.8 | 9.3 | 3.5 | 3.8 | 90.9 | 0.50 | 22.6 | 45.1 |
| | Avg. | 26.7 | 8.9 | 3.9 | 3.9 | | 0.57 | 23.2 | 40.9 |
| (Sato | 2019 | 23.4 | 5.7 | 5.0 | 2.0 | | 0.65 | 22.2 | 33.9 |
| Nishiki) | 2020 | 21.8 | 5.7 | 3.0 | 2.0 | | 0.55 | 18.5 | 33.7 |
| | 2021 | 22.3 | 6.2 | 4.0 | 1.5 | 77.9 | 0.59 | 20.3 | 34.3 |
| | Avg. | 22.5 | 5.9 | 4.0 | 1.8 | | 0.60 | 20.3 | 34.0 |

The incidence of cold damage, frost damage, and disease and pestilence was also determined. There was no incidence of cold damage during test period. According to the results of Fukagawa City and Mashike Town, the frost damage was more than that of ‘Sato Nishiki’ and equivalent to or less than ‘Nanyo’ and ‘Benishuho’.

‘Himari’ offers a combination of superior properties.

Cherry is one of the main products of fruit trees in Hokkaido. The main cultivated varieties in Hokkaido are ‘Hokko’, medium-maturing and with strong cold hardiness, and ‘Nanyo’, late-maturing and large in size, in addition to medium-maturing ‘Sato Nishiki’, which occupies about 50% of the cultivated area. Although each of these varieties has excellent characteristics, there are many points to be improved. Among them, ‘Nanyo’ and ‘Sato Nishiki’ are cross-incompatible because they have the same S genotype, and they are unstable factors of fruit production because they do not function as pollinating trees. ‘Nanyo’ has problems in the quality of fruit, such as difficulty in skin coloration and inferior transportability due to soft flesh.

‘HC10’ is a late-maturing line which is harvested in mid- to late-July in Hokkaido. Compared to ‘Nanyo’, ‘HC10’ has a large size, good skin coloration, firm flesh, and excellent eating quality. Therefore, its selling price is expected to be equal to or higher than ‘Nanyo’. The S genotype is S₁S_{3,} which is cross-compatible with most cultivated varieties including ‘Sato Nishiki’. Therefore, ‘HC10’ can improve the pollination environment of orchards and contribute to ensuring the fruiting of other varieties.

As described above, by replacing ‘HC10’ with most of ‘Nanyo’ and spreading the use of ‘HC10’, it is possible to improve the quality of Hokkaido-made cherries, contribute to the stable production of ‘Sato Nishiki’, which is a basic variety as a pollination tree, and contribute to the promotion of cherry cultivation in Hokkaido by contributing to the diversification of products not only in market shipment but also in tourist fruit farms and direct sales depots.

What is claimed is:

1. A new and distinct variety of cherry tree having the characteristics substantially as described and illustrated herein.
